(19) United States
(12) Patent Application Publication
(10) Pub. No.: US 2025/0213850 A1
(43) Pub. Date: Jul. 3, 2025

(54) Using One or More Bands to Hold a Plurality of Electrode Assemblies Against a Subject's Body

(71) Applicant: Novocure GmbH, Root D4 (CH)

(72) Inventor: Yoram WASSERMAN, Haifa (IL)

(73) Assignee: Novocure GmbH, Root D4 (CH)

(21) Appl. No.: 19/003,434

(22) Filed: Dec. 27, 2024

Related U.S. Application Data
(60) Provisional application No. 63/616,184, filed on Dec. 29, 2023.

(51) Int. Cl. A61N 1/04; A61N 1/36

(52) U.S. Cl. CPC A61N 1/0484; A61N 1/36002

(57) ABSTRACT

Alternating electric fields can be applied to a target region in a subject's body using an apparatus that comprises a plurality of electrode assemblies and one or more stretchable bands. Each of the of electrode assemblies is configured to impose an alternating electric field in the target region of the subject's body. The one or more stretchable bands are connected to the plurality of electrode assemblies, and are collectively shaped and dimensioned to press the plurality of electrode assemblies against respective locations of the subject's body so that the target region will be positioned between the plurality of electrode assemblies.

### CROSS REFERENCE TO RELATED APPLICATIONS

This application claims the benefit of U.S. Provisional Application 63/616,184, filed Dec. 29, 2023, which is incorporated herein by reference in its entirety.

### BACKGROUND

Tumor Treating Fields (TTFields) therapy is a proven approach for treating tumors using alternating electric fields at frequencies e.g., between 50 kHz-5 MHz, more commonly 100-500 KHz. The alternating electric fields are induced by electrode assemblies (e.g., arrays of capacitively coupled electrodes, also called transducer arrays) placed on the subject's skin on opposite sides of the subject's body. When an AC voltage is applied between opposing electrode assemblies, an AC current is coupled through the electrode assemblies and into the subject's body.

In the prior art, the electrode assemblies are held in place by a flexible backing that is attached to the outer surfaces of the electrode assemblies. Self-adhesive portions of the flexible backing extend laterally beyond the electrode assemblies and adhere to the subject's skin, which holds the electrode assemblies against the subject's body. But because the electrode assemblies must remain on the subject's body for extended periods of time, the self-adhesive surfaces can sometimes irritate the subject's skin, which can cause discomfort and even reduce patient compliance.

### SUMMARY OF THE INVENTION

One aspect of the invention is directed to a first apparatus for applying an alternating electric field to a target region in a subject's body. The first apparatus comprises a plurality of electrode assemblies and one or more stretchable bands. Each of the of electrode assemblies has a respective width and is configured to impose an alternating electric field in the target region of the subject's body. The one or more stretchable bands are connected to the plurality of electrode assemblies, and are collectively shaped and dimensioned to press the plurality of electrode assemblies against respective locations of the subject's body so that the target region will be positioned between the plurality of electrode assemblies.

In some embodiments of the first apparatus, the one or more stretchable bands are connected to the plurality of electrode assemblies at corresponding locations on the one or more stretchable bands, and have widths at the corresponding locations that match the respective ones of the plurality of electrode assemblies within ±1 cm or, in some embodiments, within ±0.5 cm.

In some embodiments of the first apparatus, the widths of all the electrode assemblies are substantially the same, and the one or more stretchable bands have a substantially uniform width.

In some embodiments of the first apparatus, at least one of the one or more stretchable bands is positioned both over at least one of the plurality of electrode assemblies and between at least one adjacent pair of electrode assemblies.

In some embodiments of the first apparatus, the one or more stretchable bands are constructed from a breathable material to allow perspiration formed at the subject's skin to pass through the one or more stretchable bands.

In some embodiments of the first apparatus, at least one stretchable band, from the one or more stretchable bands, presses against at least one electrode assembly, from the plurality of electrode assemblies, at a portion of the at least one stretchable band that excludes the opposite longitudinal ends of the at least one stretchable band.

In some embodiments of the first apparatus, a layer of conductive adhesive or a layer of conductive hydrogel is disposed on a front surface of each of the electrode assemblies. In some embodiments of the first apparatus, each of the electrode assemblies comprises at least one conductive pad, and a layer of dielectric material is positioned directly in front of the at least one conductive pad.

In some embodiments of the first apparatus, each of the electrode assemblies comprises at least one conductive pad, and a layer of graphite is positioned in front of the at least one conductive pad. Optionally, in these embodiments, a layer of conductive adhesive or a layer of conductive hydrogel is positioned against both a front side of the layer of graphite and a rear side of the layer of graphite.

In some embodiments of the first apparatus, each of the plurality of electrode assemblies includes a first subassembly that is removably connectable to a second subassembly, and the first subassembly comprises a first conductive layer with a front surface facing toward the subject's body and a rear surface facing away from the subject's body wherein the front surface of the first conductive layer is a front surface of the first subassembly. The first subassembly further includes a dielectric layer positioned between a rear of the first subassembly and the rear surface of the first conductive layer, and one or more electrode elements positioned between the rear of the first subassembly and the dielectric layer. The second subassembly comprises at least a second conductive layer.

Another aspect of the invention is directed to a second apparatus for applying an alternating electric field to a target region in a subject's body. The second apparatus comprises a plurality of electrode assemblies and one or more stretchable bands. Each of the of electrode assemblies has a respective width and is configured to impose an alternating electric field in the target region of the subject's body. The one or more stretchable bands are connected to the plurality of electrode assemblies, and are collectively shaped and dimensioned to press the plurality of electrode assemblies against respective locations of the subject's body so that the target region will be positioned between the plurality of electrode assemblies. In the second apparatus, the one or more stretchable band is a single stretchable band and is configured to form a closed loop when a first end of the single stretchable band is attached to a second end of the single stretchable band.

In some embodiments of the second apparatus, an unstretched length of the closed loop is adjustable.

In some embodiments of the second apparatus, the single stretchable band has a first end and a second end, and the apparatus further comprises a first fastener positioned at the first end of the single stretchable band and a second fastener positioned at the second end of the single stretchable band. The first and second fasteners are configured and positioned to hold the single stretchable band in a closed loop shape when the first and second fasteners mate with each other.

Optionally, in the embodiments described in the previous paragraph, the first and second fasteners are hook-and-loop fasteners or are reversible reclosable fasteners. Optionally, in the embodiments described in the previous paragraph, either the first fastener, or the second fastener, or both, comprise a layer of adhesive.

Some embodiments of the second apparatus further comprise a plurality of attachment structures, each of which is disposed at a respective location on the single stretchable band, and each of the attachment structures is attached to a respective one of the electrode assemblies.

Some embodiments of the second apparatus further comprise a plurality of first fasteners disposed on the single stretchable band and at least one second fastener disposed on each of the electrode assemblies, and each of the second fasteners is mated with a respective one of the plurality of first fasteners. In these embodiments, the first and second fasteners are hook-and-loop fasteners or are reversible reclosable fasteners.

In some embodiments of the second apparatus, at least one of (a) a plurality of front portions of the single stretchable band that faces the subject's body and (b) at least a portion of a rear surface of each of the electrode assemblies that faces away from the subject's body is coated with an adhesive that is configured to adhere respective front portions of the single stretchable band to respective rear surfaces of the plurality of electrode assemblies.

Another aspect of the invention is directed to a third apparatus for applying an alternating electric field to a target region in a subject's body. The third apparatus comprises a plurality of electrode assemblies and one or more stretchable bands. Each of the of electrode assemblies has a respective width and is configured to impose an alternating electric field in the target region of the subject's body. The one or more stretchable bands are connected to the plurality of electrode assemblies, and are collectively shaped and dimensioned to press the plurality of electrode assemblies against respective locations of the subject's body so that the target region will be positioned between the plurality of electrode assemblies. In the third apparatus, at least one of the one or more stretchable bands is positioned only between adjacent pairs of electrode assemblies.

In some embodiments of the third apparatus, the at least one of the one or more stretchable bands is connected to at least one of the plurality of electrode assemblies via a hook-and-loop fastener or by a reversible reclosable fastener. In some embodiments of the third apparatus, the at least one of the one or more stretchable bands is sewn onto a support of at least one of the plurality of electrode assemblies. In some embodiments of the third apparatus, the at least one of the one or more stretchable bands is connected to at least one of the plurality of electrode assemblies by means of an adhesive.

In some embodiments of the third apparatus, each of the one or more stretchable bands is positioned only between adjacent pairs of electrode assemblies such that each pair of electrode assemblies are connected by a stretchable band. Optionally, in these embodiments, alternating pairs of stretchable bands and electrode assemblies are linked together into a closed loop. Optionally, in these embodiments, an unstretched length of the closed loop is adjustable.

Another aspect of the invention is directed to a kit for use in applying an alternating electric field to a target region in a subject's body. The kit includes a plurality of electrode assemblies, each of which has a respective width and each of which is configured to impose an alternating electric field in the target region of the subject's body. The kit also includes one or more stretchable bands connecting two adjacent electrode assemblies or connecting a series of electrode assemblies and are collectively shaped and dimensioned to press the plurality of electrode assemblies against respective locations of the subject's body so that the target region will be positioned between the plurality of electrode assemblies.

Another aspect of the invention is directed to a first method of applying an alternating electric field to a target region in a subject's body. The first method comprises positioning a plurality of electrode assemblies at respective locations of the subject's body so that the target region is positioned between the plurality of electrode assemblies. Each of the electrode assemblies has a respective width. The first method also comprises effecting one or more forces that press the plurality of electrode assemblies onto the respective locations of the subject's body using one or more stretchable bands connecting two adjacent electrode assemblies or connecting a series of electrode assemblies; and applying an AC voltage between one or more pairs of the plurality of electrode assemblies to impose the alternating electric field in the target region. The AC voltage has a frequency between 50 kHz and 1 MHz.

In some instances of the first method, at least one of the one or more

stretchable bands is positioned both over at least one of the plurality of electrode assemblies and between at least one adjacent pair of electrode assemblies. In some instances of the first method, at least one of the one or more stretchable bands is positioned over at least one of the plurality of electrode assemblies. In some instances of the first method, at least one of the one or more stretchable bands is positioned only between adjacent pairs of electrode assemblies. In some instances of the first method, each of the one or more stretchable bands is positioned only between adjacent pairs of electrode assemblies such that each pair of electrode assemblies are connected by a stretchable band. In some instances of the first method, the one or more stretchable bands have a width that matches the width of each electrode assembly, respectively, within ±1 cm. In some instances of the first method, the AC voltage has a frequency between 100 kHz and 300 kHz.

Another aspect of the invention is directed to a second method of applying an alternating electric field to a target region in a subject's body. The second method comprises positioning a plurality of electrode assemblies at respective locations of the subject's body so that the target region is positioned between the plurality of electrode assemblies. Each of the electrode assemblies has a respective width. The second method also comprises effecting one or more forces that press the plurality of electrode assemblies onto the respective locations of the subject's body using one or more straps comprising a hook and loop fastener and/or reversible reclosable fastener connecting two adjacent electrode assemblies or connecting a series of electrode assemblies; and applying an AC voltage between one or more pairs of the plurality of electrode assemblies to impose the alternating electric field in the target region. The AC voltage has a frequency between 50 kHz and 1 MHz.

In some instances of the second method, at least one of the one or more straps comprising a hook and loop fastener and/or reversible reclosable fastener is positioned both over at least one of the plurality of electrode assemblies and between at least one adjacent pair of electrode assemblies. In some instances of the second method, at least one of the one or more straps comprising a hook and loop fastener and/or reversible reclosable fastener is positioned over at least one of the plurality of electrode assemblies. In some instances of the second method, at least one of the one or more straps comprising a hook and loop fastener and/or reversible reclosable fastener is positioned only between adjacent pairs of electrode assemblies. In some instances of the second method, each of the one or more straps comprising a hook and loop fastener and/or reversible reclosable fastener is positioned only between adjacent pairs of electrode assemblies such that each pair of electrode assemblies are connected by a strap comprising a hook and loop fastener and/or reversible reclosable fastener. In some instances of the second method, the one or more straps comprising a hook and loop fastener and/or reversible reclosable fastener have a width that matches the width of each electrode assembly, respectively, within ±1 cm. In some instances of the second method, the AC voltage has a frequency between 100 kHz and 300 kHz.

Another aspect of the invention is directed to a third method of applying an alternating electric field to a target region in a subject's body. The third method comprises positioning a plurality of electrode assemblies at respective locations of the subject's body so that the target region is positioned between the plurality of electrode assemblies. Each of the electrode assemblies has a respective width. The third method also comprises effecting one or more forces that press the plurality of electrode assemblies onto the respective locations of the subject's body using one or more stretchable bands contacting two adjacent electrode assemblies or contacting a series of electrode assemblies; and applying an AC voltage between one or more pairs of the plurality of electrode assemblies to impose the alternating electric field in the target region. The AC voltage has a frequency between 50 kHz and 1 MHz.

In some instances of the third method, at least one of the one or more stretchable bands is positioned both over at least one of the plurality of electrode assemblies and between at least one adjacent pair of electrode assemblies. In some instances of the third method, at least one of the one or more stretchable bands is positioned over at least one of the plurality of electrode assemblies. In some instances of the third method, a single stretchable band is positioned over each one of the plurality of electrode assemblies. In some instances of the third method, at least one of the one or more stretchable bands is positioned only between adjacent pairs of electrode assemblies. In some instances of the third method, each of the one or more stretchable bands is positioned only between adjacent pairs of electrode assemblies such that each pair of electrode assemblies are connected by a stretchable band. In some instances of the third method, the one or more stretchable bands have a width that matches the width of each electrode assembly, respectively, within ±1 cm. In some instances of the third method, the AC voltage has a frequency between 100 kHz and 300 kHz.

Another aspect of the invention is directed to a fourth method of applying an alternating electric field to a target region in a subject's body. The fourth method comprises positioning a plurality of electrode assemblies at respective locations of the subject's body so that the target region is positioned between the plurality of electrode assemblies. Each of the electrode assemblies has a respective width. The fourth method also comprises effecting one or more forces that press the plurality of electrode assemblies onto the respective locations of the subject's body using one or more straps comprising a hook and loop fastener and/or reversible reclosable fastener contacting two adjacent electrode assemblies or contacting a series of electrode assemblies; and applying an AC voltage between one or more pairs of the plurality of electrode assemblies to impose the alternating electric field in the target region. The AC voltage has a frequency between 50 kHz and 1 MHz.

In some instances of the fourth method, at least one of the one or more straps comprising a hook and loop fastener and/or reversible reclosable fastener is positioned both over at least one of the plurality of electrode assemblies and between at least one adjacent pair of electrode assemblies. In some instances of the fourth method, at least one of the one or more straps comprising a hook and loop fastener and/or reversible reclosable fastener is positioned over at least one of the plurality of electrode assemblies. In some instances of the fourth method, a single strap comprising a hook and loop fastener and/or reversible reclosable fastener is positioned over each one of the plurality of electrode assemblies. In some instances of the fourth method, at least one of the one or more straps comprising a hook and loop fastener and/or reversible reclosable fastener is positioned only between adjacent pairs of electrode assemblies. In some instances of the fourth method, each of the one or more straps comprising a hook and loop fastener and/or reversible reclosable fastener is positioned only between adjacent pairs of electrode assemblies such that each pair of electrode assemblies are connected by a strap comprising a hook and loop fastener and/or reversible reclosable fastener. In some instances of the fourth method, the one or more straps comprising a hook and loop fastener and/or reversible reclosable fastener have a width that matches the width of each electrode assembly, respectively, within ±1 cm. In some instances of the fourth method, the AC voltage has a frequency between 100 kHz and 300 kHz.

Another aspect of the invention is directed to a fourth apparatus for applying an alternating electric field to a target region in a subject's body. The fourth apparatus comprises a plurality of electrode assemblies and one or more straps. Each of the electrode assemblies is configured to impose an alternating electric field in the target region of the subject's body. The one or more straps are collectively shaped and dimensioned to hold the plurality of electrode assemblies against respective locations of the subject's body so that the target region will be positioned between the plurality of electrode assemblies. The one or more straps and the plurality of electrode assemblies are collectively shaped and dimensioned to completely surround a portion of the subject's body.

In some embodiments of the fourth apparatus, the one or more straps and the plurality of electrode assemblies are collectively held together using one or more hook-and-loop fasteners and/or reversible reclosable fasteners. Optionally, in these embodiments, there is only a single strap configured to form a closed loop around the subject's body and hold the electrode assemblies against the respective locations of the subject's body.

In some embodiments of the fourth apparatus, at least one of the one or more straps is positioned both over at least one of the plurality of electrode assemblies and between at least one adjacent pair of electrode assemblies. In some embodiments of the fourth apparatus, at least one of the one or more straps is positioned only between adjacent pairs of electrode assemblies.

In some embodiments of the fourth apparatus, each of the one or more straps is positioned only between adjacent pairs of electrode assemblies such that each pair of electrode assemblies are connected by a strap. Optionally, in these embodiments, there are either two electrode assemblies and two straps, or four electrode assemblies and four straps, in each case configured to form a closed loop around the subject's body.

Another aspect of the invention is directed to a fifth apparatus for applying an alternating electric field to a target region in a subject's body. The fifth apparatus comprises a plurality of electrode assemblies and one or more stretchable bands. Each of the electrode assemblies has a respective width and each is configured to impose an alternating electric field in the target region of the subject's body. The one or more stretchable bands are connected to the plurality of electrode assemblies, and are collectively shaped and dimensioned to press the plurality of electrode assemblies against respective locations of the subject's body so that the target region will be positioned between the plurality of electrode assemblies. The one or more stretchable bands that are connected to respective electrode assemblies have a width that matches the width of the respective electrode assembly within ±1 cm.

In some embodiments of the fifth apparatus, the widths of all the electrode assemblies are substantially the same, and the one or more stretchable bands have a substantially uniform width. In some embodiments of the fifth apparatus, at least one of the one or more stretchable bands is positioned both over at least one of the plurality of electrode assemblies and between at least one adjacent pair of electrode assemblies.

Another aspect of the invention is directed to a sixth apparatus for applying an alternating electric field to a target region in a subject's body. The sixth apparatus comprises a plurality of electrode assemblies and one or more stretchable bands. Each of the electrode assemblies has a respective width and each is configured to impose an alternating electric field in the target region of the subject's body. The one or more stretchable bands are connected to the plurality of electrode assemblies, and are collectively shaped and dimensioned to press the plurality of electrode assemblies against respective locations of the subject's body so that the target region will be positioned between the plurality of electrode assemblies. The one or more stretchable bands that are connected to respective electrode assemblies have a width that matches the width of the respective electrode assembly within ±1 cm. In the sixth apparatus, the one or more stretchable band is a single stretchable band and is configured to form a closed loop when a first end of the single stretchable band is attached to a second end of the single stretchable band.

In some embodiments of the sixth apparatus, an unstretched length of the closed loop is adjustable.

In some embodiments of the sixth apparatus, the single stretchable band has a first end and a second end, and the apparatus further comprises a first fastener positioned at the first end of the single stretchable band and a second fastener positioned at the second end of the single stretchable band. In these embodiments, the first and second fasteners are configured and positioned to hold the single stretchable band in a closed loop shape when the first and second fasteners mate with each other.

Optionally, in the embodiments described in the previous paragraph, the first and second fasteners are hook-and-loop fasteners or are reversible reclosable fasteners. Optionally, in the embodiments described in the previous paragraph, either the first fastener, or the second fastener, or both, comprise a layer of adhesive.

Some embodiments of the sixth apparatus further comprise a plurality of attachment structures, each of which is disposed at a respective location on the single stretchable band, and each of the attachment structures is attached to a respective one of the electrode assemblies.

Some embodiments of the sixth apparatus further comprise a plurality of first fasteners disposed on the single stretchable band and at least one second fastener disposed on each of the electrode assemblies. In these embodiments, each of the second fasteners is mated with a respective one of the plurality of first fasteners, and the first and second fasteners are hook-and-loop fasteners or are reversible reclosable fasteners.

In some embodiments of the sixth apparatus, at least one of (a) a plurality of front portions of the single stretchable band that faces the subject's body and (b) at least a portion of a rear surface of each of the electrode assemblies that faces away from the subject's body is coated with an adhesive that is configured to adhere respective front portions of the single stretchable band to respective rear surfaces of the plurality of electrode assemblies.

Another aspect of the invention is directed to a seventh apparatus for applying an alternating electric field to a target region in a subject's body. The seventh apparatus comprises a plurality of electrode assemblies and one or more stretchable bands. Each of the electrode assemblies has a respective width and each is configured to impose an alternating electric field in the target region of the subject's body. The one or more stretchable bands are connected to the plurality of electrode assemblies, and are collectively shaped and dimensioned to press the plurality of electrode assemblies against respective locations of the subject's body so that the target region will be positioned between the plurality of electrode assemblies. The one or more stretchable bands that are connected to respective electrode assemblies have a width that matches the width of the respective electrode assembly within ±1 cm. In the seventh apparatus, at least one of the one or more stretchable bands is positioned only between adjacent pairs of electrode assemblies.

In some embodiments of the seventh apparatus, the at least one of the one or more stretchable bands is connected to at least one of the plurality of electrode assemblies via a hook-and-loop fastener or by a reversible reclosable fastener. In some embodiments of the seventh apparatus, the at least one of the one or more stretchable bands is sewn onto a support of at least one of the plurality of electrode assemblies. In some embodiments of the seventh apparatus, the at least one of the one or more stretchable bands is connected to at least one of the plurality of electrode assemblies by means of an adhesive. In some embodiments of the seventh apparatus, each of the one or more stretchable bands is positioned only between adjacent pairs of electrode assemblies such that each pair of electrode assemblies are connected by a stretchable band.

In some embodiments of the seventh apparatus, each of the one or more stretchable bands are positioned only between adjacent pairs of electrode assemblies such that each pair of electrode assemblies are connected by a stretchable band, and alternating pairs of stretchable bands and electrode assemblies are linked together into a closed loop. Optionally, in these embodiments, an unstretched length of the closed loop is adjustable.

### BRIEF DESCRIPTION OF THE DRAWINGS

FIGS. 1A-1B are top views of a stretchable band with electrode assemblies disposed thereon.

FIGS. 2A-2B are plan views of a plurality of stretchable band sections with one or more electrode assemblies disposed thereon.

FIGS. 3A-3B are plan views of an arrangement of electrode assemblies and straps linked together.

FIGS. 4A-4D depict a subject wearing an arrangement of four electrode assemblies linked together using multiple bands.

FIG. 5 is a cross sectional diagram of one embodiment of an electrode assembly that can be held in position by one or more bands.

FIG. 6 is a cross sectional diagram of another embodiment of an electrode assembly that can be held in position by one or more bands.

FIG. 7 is a schematic representation of a system that includes electrode assemblies placed on a subject and secured by a stretchable band, and an AC generator and controller to controllably apply TTFields to a target region within a subject's body.

FIG. 8 is a flowchart of an example procedure for applying an alternating electric field to a target region in a subject's body.

Various embodiments are described in detail below with reference to the accompanying drawings, wherein like reference numerals represent like elements.

### DESCRIPTION OF THE PREFERRED EMBODIMENTS

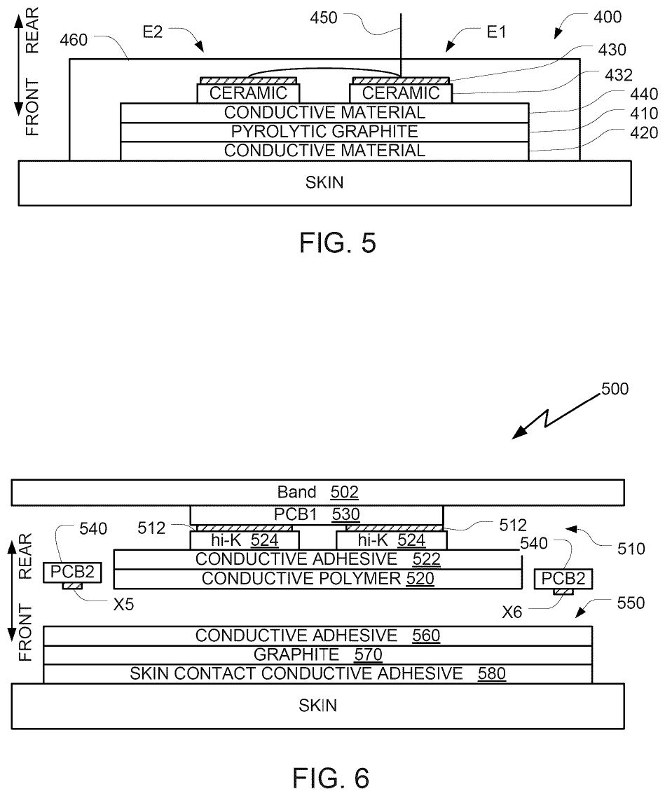

The present disclosure is directed to systems, apparatus, methods, and other implementations in which one or more stretchable bands (or, in some embodiments, straps) are used for securing electrode assemblies against a subject's body. These electrode assemblies are used for applying alternating electric fields to a target region in the subject's body. Note that in the descriptions below, the front of any given electrode assembly is the side that faces the subject's body, and the rear of any given electrode assembly is the side that faces away from the subject's body (e.g., as depicted in FIGS. 5-6).

The stretchable bands can be used in a number of placement configurations. In one example, a single stretchable band (i.e., a band that can resiliently be expanded during use to fit specific dimensions of a body part of a particular subject) can be used to press a plurality of electrode assemblies on to the body part when the band is wrapped around the body part surrounding the target region. In a second example, multiple band sections (either resiliently expandable or unstretchable) can be linked into a chain that surrounds the body part and presses a plurality of electrode assemblies against the subject's body. In a third example, a chain of linked sections and one or more electrode assemblies, i.e., at least one electrode assembly linked to adjacent band sections, is used for surrounding a body part with electrode assemblies.

Any of these configurations can be used to implement an apparatus for applying an alternating electric field to a target region in a subject's body that includes a plurality of electrode assemblies and one or more stretchable bands. Each of the electrode assemblies (also referred to as transducer arrays), has a respective width and is configured to impose an alternating electric field in the target region of the subject's body. The one or more stretchable bands are connected to the plurality of electrode assemblies. The one or more stretchable bands are collectively shaped and dimensioned to press the plurality of electrode assemblies against respective locations of the subject's body so that the target region will be positioned between the plurality of electrode assemblies on the body.

The stretchable/flexible characteristics of the band (or bands) not only allow for use with body parts of different dimensions and contours, but operate to press the electrode assemblies against the subject's body. Additionally, using stretchable bands/straps can be less time consuming than having to affix bandages (or other types of fastening or securing structures) to the electrode assemblies and the subject's skin. Using stretchable bands can also allow for a wider choice of materials (e.g., breathable materials, e.g., cotton fabrics, through which sweat can permeate) that can be more pleasant, from aesthetic and comfort perspectives, to the subjects. Furthermore, a stretchable band or form-fitting adjustable strap that provides a pressing force on the electrode assemblies on the body may obviate the need for an overlay bandage with an aggressive adhesive to secure the electrode assemblies in place. This in turn may improve compliance in a subject's duration of treatment and reduce skin irritation from repeated removal and replacement of the electrode assemblies.

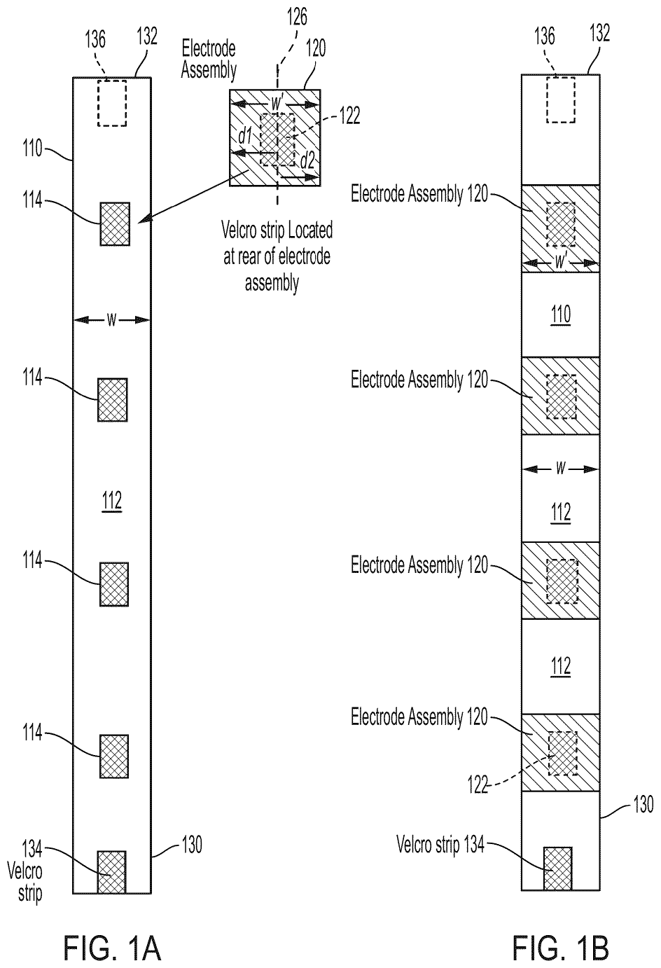

FIGS. 1A and 1B are plan views of an embodiment that relies on a single stretchable band to hold a plurality of electrode assemblies in place on a subject's body. In FIG. 1A, a stretchable band 110 is depicted without electrode assemblies placed or secured onto it. In FIG. 1B, the stretchable band 110 is depicted with a plurality of electrode assemblies attached to its contact surface 112 (i.e., the surface that contacts the subject's skin).

In FIGS. 1A and 1B, the stretchable band 110 (or, in some embodiments, a strap) is shown as being a rectangular strip that has a substantially uniform width, marked as w. It is noted, however, that in various embodiments the stretchable band need not have such a rectangular shape, but rather may have other shapes and dimensions suitable to have a plurality of electrode assemblies disposed thereon. The stretchable band, for example, may have an irregular shape (e.g., wavy or uneven edges). As also shown in FIG. 1A and in FIG. 1B, an electrode assembly 120 (as well as the other electrode assemblies 120 of FIGS. 1A-B) has a width w′.

Although FIGS. 1A and 1B depict the example electrode assembly 120 as having a substantially uniform width, other embodiments of the electrode assemblies (including embodiments in which the electrodes are formed on printed circuit boards with irregular shapes) may have non-uniform dimensions. For example, with the electrode assembly width, as measured from the longitudinal axis of the band, moving orthogonally towards the points on the outside edges of the electrode assembly, may vary at different points along the longitudinal axis of the electrode assembly. More generally, for a regularly and irregularly shaped electrode assembly, the width w′ associated with the electrode assembly such as the assembly 120 may be deemed to be the sum of a first orthogonal distance, d1, from the farthest edge point on one side of the electrode to a longitudinal axis 126 of the electrode assembly, and a second orthogonal distance, d2, from the farthest edge point on the opposite side (with respect to the longitudinal axis 126) of the electrode assembly to the longitudinal axis 126.

In some embodiments, the width w of the stretchable band 110 matches the width w′ of each of the electrode assemblies 120 that is positioned on the stretchable band 110. The matching between w and w′ can be, for example, within ±5 cm, within ±2 cm, within ±1.5 cm, within ±1 cm, within ±8 mm, within ±7 mm, within ±6 mm, within ±5 mm, within ±4 mm, within ±3 mm, within ±2 mm, within ±1 mm, within ±0.8 mm, or within ±0.5 mm. When the width of an electrode assembly matches (within a tolerance value) the width of the stretchable band (at least at a position on the stretchable band where the electrode assembly is placed), the force exerted by the stretchable band on the electrode assembly is more uniformly distributed on the electrode assembly, thus allowing the relative position of the electrode assembly relative to the target region to be more easily maintained.

In the example depicted in FIG. 1B, all of the electrode assemblies 120 have the same width, and the band 110 has a uniform width that matches (within a tolerance) the widths of all the electrode elements. But in alternative embodiments, some of the electrode assemblies 120 could have different widths. In these embodiments, the portions of the band 110 that provide support to the narrower electrode elements can be narrower, and the portions of the band 110 that provide support to the wider electrode elements can be wider.

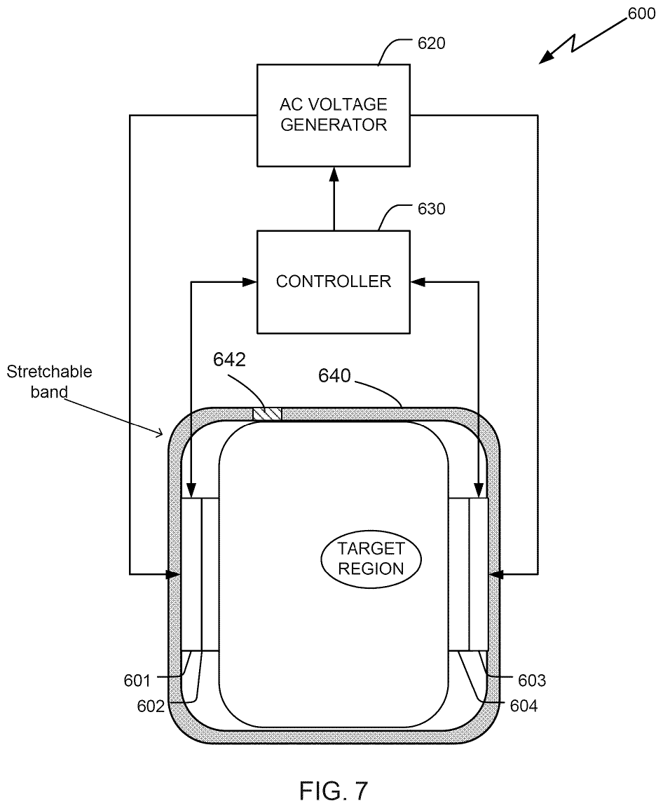

In embodiments of FIGS. 1A and 1B, in which a single stretchable band (in this example, the stretchable band 110) is used, the stretchable band 110 forms a closed loop when a first end 130 of the band 110 is attached to a second end 132 of the band. An example of this is depicted in FIG. 7, in which a single stretchable band is attached at its ends to form a closed loop that presses against two electrode assemblies positioned at opposite sides of a target region in a subject's body. Returning now to FIG. 1, the stretchable band 110 may include fasteners configured to attach the two ends 130 and 132 to form a closed loop. For example, the stretchable band 110 may include a first fastener 134 positioned at the first end 130 of the single stretchable band, and a second companion fastener 136 positioned at the second end of the single stretchable band.

The first and second fasteners 134 and 136 are configured and positioned to hold the single stretchable band in a closed loop shape when the first and second fasteners mate with each other. The first and second fasteners 134 and 136 may be hook-and-loop fasteners (for example, the first fastener may comprise hook material and the second fastener may comprise loop material, or vice-versa), reversible reclosable fasteners such as those fasteners that rely on interlocking mushroom-shaped heads, or some other type of fasteners. In the example of FIG. 1, the two fasteners 134 and 136 are shown as hook-and-loop fasteners (e.g., Velcro®, Velcro USA, Inc., Manchester, NH, USA), but, as noted, other types of fasteners for attaching the two ends into a closed loop (e.g., a belt-type buckle, reversible reclosable fasteners, hook and eye closures, ring, slide and hook closures, etc.) may be used. Examples of reversible reclosable fasteners include DUAL-LOCK™ reclosable fastener provided by 3M™ of Minneapolis, MN, USA; or ALFA-LOK™ (Velcro USA, Inc., Manchester, NH, USA).

It is to be noted that in FIGS. 1A-B the fastener 136 is depicted on an opposite surface of the stretchable band 110 than the surface on which the fastener 134 is positioned (and, similarly, fasteners 136 and 134 could be on opposite surfaces of the stretchable band 110 to that shown in FIGS. 1A-B. This allows attachment of the two fasteners 134 and 136 without having to twist one of the ends so as to allow the two hook-and-loop fasteners to mate. To attach the fasteners to the stretchable band (i.e., during construction of the stretchable band), one or both of the fasteners may include an adhesive layer, disposed on a surface of the fastener(s) that is to be secured to the surfaces of the stretchable band 110 proximate the ends 130 and 132 of the band 110. Examples of suitable adhesive that can be used to attach the fasteners to the stretchable band 110 include acrylic adhesive, cyanoacrylate adhesive, epoxy adhesive, structural acrylic adhesive, hot-melt adhesive, adhesive foam, etc. Thus, in the example of hook-and-loop fasteners, the adhesive layer on the rear of a hook-and-loop strip (i.e., the smooth side without the hooks or loops structures) of the first fastener 134 is affixed to the surface of the stretchable band 110 on which the electrode assemblies are positioned, while an adhesive layer at the rear of the fastener 136 is attached to the opposite (hidden-shown in dotted line) surface of the stretchable band 110 shown in FIGS. 1A-B. Other means of attachment of the fasteners to the stretchable band 110 may also be used, for example, Velcro® strips may be stitched/sewn onto the ends of the stretchable band.

In various examples, attaching the two ends 130 and 132 together can be performed so as to adjust the unstretched length of the closed loop. For example, when using a hook-and-loop type fasteners, one (or both) of the fasteners can have a length(s) (parallel to a longitudinal axis of the stretchable band 110) that allows the mating of one fastener, e.g., 134, with the fastener 136 at different positions along the length of fastener 136, thus allowing the end 130 of the band 110 to be attached to the fastener 136 at an adjustable distance from the end 132 of the stretchable band 110 (for example, according to the chosen amount of overlap of the fastener surfaces). In other examples, the adjustment of the unstretched length of the closed loop formed by attaching the two ends of the band 110 can be facilitated by an adjustment mechanism, such as a buckle through which one of the ends of the stretchable band can be wrapped in order to control the length of the unstretched closed loop formed by the band.

Placement of electrode assemblies against the subject's body can be performed, for example, by positioning the electrode assemblies on the subject's body (optionally using a biocompatible adhesive to at least temporarily ensure that the electrode assemblies remain stationary when placed on the body), and then placing the stretchable band on top of the electrode assemblies, and attaching the ends of the band (possibly adjusting the length of the band) to create sufficient tension to press on the electrode assemblies so that the electrode assemblies do not move during delivery of the treatment. Although stretching a band laterally outward from the point of contact with an electrode assembly provides only minimal force in the perpendicular direction to push the electrode assembly onto the subject's skin, because the band wraps around the body part, the tension in the band also provides a force in the perpendicular direction to push the electrode assembly onto the subject's skin. Attachment structures can be used to securely attach the electrode assemblies to the stretchable band. For example, the stretchable band can include a plurality of attachment structures, each of which is disposed at a respective location on the single stretchable band, with each of the attachment structures being attachable to a respective one of the electrode assemblies.

In the example depicted in FIG. 1A, the attachment structures are hook-and-loop (e.g., Velcro®) strips 114 that are disposed at respective locations on the stretchable band 110. Each of the electrode assemblies may include a counterpart fastener, such as a fastener 122 (which may also be a hook-and-loop fastener) of electrode assembly 120, to mate with a respective one of the attachment structures 114. The locations on the stretchable band 110 where the attachment structures 114 are placed may have been pre-determined to correspond to locations to which the counterpart fasteners of the electrode assemblies attach so that when the stretchable band 110 and the electrode assemblies disposed thereon are placed on the subject's body part, the electrode assemblies are arranged in a suitable configuration to deliver the TTFields treatment to the target location in the subject's body.

Other types of attachment structures (and their corresponding counterpart fasteners) may be used in place of the hook-and-loop fasteners described above. For example, the first fastener 134 at the first end and the second fastener 136 at the second end may be reversible reclosable fasteners (for example, fasteners that rely on interlocking mushroom-shaped heads). And the attachment structures 114 and the fasteners 122 attached to the electrode assemblies 120 may be reversible reclosable fasteners. In another example, the fasteners to attach the electrode assemblies to the stretchable band may include depressions or receiving structures disposed at pre-determined locations on the stretchable band to which the rear portions of the electrode can be snugly fitted. In yet another example of a fastener to attach the first and second end or the electrode assemblies 120 to pre-determined positions on the stretchable band 110, the rear portions of the electrode assemblies may include an adhesive layer (optionally protected by a peelable cover or release liner that is removed just prior to attachment) that is pressed against the stretchable band at pre-determined locations on the band.

In some embodiments, the electrode assemblies may include fabric patches that can be sewn to the stretchable bands. Optionally, in embodiments in which an adhesive layer is used to attach the electrode assemblies to the stretchable band 110, a companion adhesive region may be disposed at the pre-determined locations on the stretchable band 110 to increase the strength of the adhesive bond used for attaching the electrode assemblies to the stretchable band. Thus, in such embodiments, at least one of (a) a plurality of front portions of the single stretchable band that faces the subject's body, and (b) at least a portion of a rear surface of each of the electrode assemblies that faces away from the subject's body, is coated with an adhesive that is configured to adhere respective front portions of the single stretchable band to respective rear surfaces of the plurality of electrode assemblies.

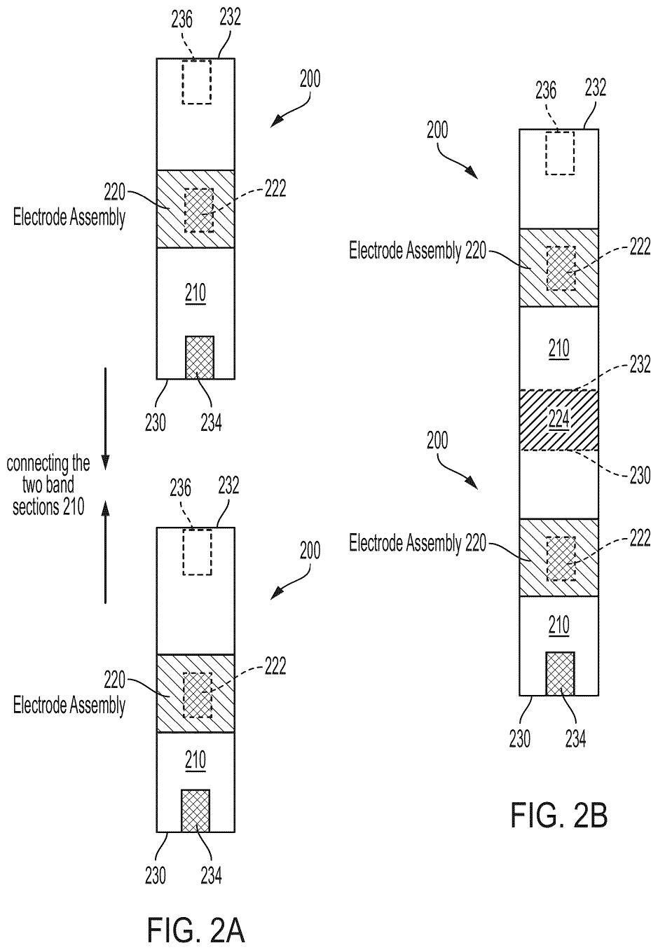

FIGS. 2A and 2B are plan views of an embodiment that relies on a plurality of modules (band sections 210) to hold a plurality of electrode assemblies in place at desired positions on a subject's body. More specifically, FIG. 2A depicts two modules 200 before they have been connected to each other, and FIG. 2B depicts those same two modules 200 after they have been connected to each other.

Each of the modules 200 includes one electrode assembly 220 and a band (or band section) 210 that extends in two opposite directions (i.e., up and down in the FIGS. 2A/2B example) from the electrode assembly. These modules 200 are connected to each other in series by connecting the end of each band 210 to the beginning of the next band 210 to form a chain of bands. Eventually, the end of the last band in the series may be connected to the beginning of the first band, which forms a closed loop (not shown).

Consider an example apparatus that includes two modules 200, as depicted in FIGS. 2A/2B. In this example, each module 200 includes an electrode assembly 220 affixed to a respective stretchable band 210. Optionally, and similarly to the stretchable band implementation depicted in FIGS. 1A and 1B, the electrode assemblies can be initially placed at the desired locations on the subject's body part (e.g., using a biocompatible adhesive layer disposed on a front portion of the electrode assemblies that causes the assemblies to adhere, at least temporarily, to the body part) so that the target region is located between a given pair (or multiple pairs) of electrode assemblies.

The electrode assemblies 220 can optionally include attachment structures disposed on part or all of the rear portions of the electrode assemblies. In the example of FIGS. 2A-B, the attachment structures 222 are shown to occupy a smaller area than the front surfaces of the electrode assemblies 220. The attachment structures may be, in some embodiments, patches of hook or loop material that mate with companion patches disposed on a contact surface (i.e., the surface of the band 210 contacting the subject's body part). For example, if a patch of hook material is disposed on the rear portions of the electrode assemblies, the stretchable bands may include patches (possibly of similar shapes and dimensions) of loop material on the contact surface of the band to mate with the hook material patches on the rears of the electrode assemblies. Another type of attachment structure may be a reversible reclosable fastener (such as a mushroom-head interlocking device).

Linking separate stretchable sections to each other in series to form a chain can be performed in various ways. For example, in the example depicted in FIGS. 2A and 2B, each of the stretchable band sections 210 may further include fasteners (e.g., fasteners 234 and 236 for each section 210) configured to securely mate with a companion fastener attached to another stretchable band section. For example, when using a hook-and-loop mechanism, the fastener 234 may be a hook fastener that can be secured to a loop-type fastener (such as the fastener 236). In the embodiments of FIGS. 2A-B, one type of fastener may be attached to one end of the band, while its companion fastener is attached at the opposite end of the band. The fastener 234 and companion fastener 236 may be on opposite surfaces of the band 210. However, unlike the embodiments of FIGS. 1A-B, each fastener mates with a companion fastener of another band. For instance, in the example of FIGS. 2A-B, the loop-type fastener 236 of the lower section 210 can be attached to the hook fastener 234 of the upper section 210, thus forming a chain of two stretchable band sections.

As can further be seen in FIG. 2B, when the band sections 210 are secured to each other, the size of the overlapping region 224 can be adjusted in order to allow adjustment in the overall length of a resultant chain so that the chain can be better fitted to the unique dimensions of a body part against which the entire chain (comprising all the sections 210 and their respective electrode assemblies 220) is placed. For example, the length of a hook-and-loop fastener patch (i.e., the side of the patch extending in the direction of the longitudinal axis of the stretchable band) of the hook-and-loop type fasteners may be made long enough such that the overlapping region, when a hook-type fastener of one section mates with a loop-type fastener of the other section, can be adjusted (e.g., not all of the entire lengths of either of the mating patches needs to be used).

In implementations that rely on a different type of fastening mechanism that does not have an overlapping region as the one shown in the diagram 202 of FIG. 2, the lengths of the resultant chain of sections can be controlled, for example, by wrapping the interlocking pieces around each other, by using a buckle to control the available length of the interlocking elements, etc.

While FIGS. 2A and 2B illustrate the linking of two modules 200 via their stretchable band sections, any number of modules (sections) (e.g., 3-10) may be used to extend the resultant linked chain. For example, in FIGS. 2A-B, another section can be affixed to the fastener located at the end 232 of the upper section 210 (at the end 230 of the lower section 210 in the figures. In the case of using hook-and-loop fasteners, and assuming that the fasteners 236 attached to the band sections are loop-type fasteners, another fastener section (new section), similar in its structure and configuration to the sections 210 of FIGS. 2A-B may be attached to one of the other sections 210 by attaching a hook-type fastener 234 at one of the ends of the new section to the loop-type fastener 236. Similarly, that new section, or another section, may be attached to the lower section 210 of FIGS. 2A-B, at the end 230 of that section, by attaching a loop-type fastener 236 secured to one of the ends of the new section, to the hook type fastener 234 disposed at the end 230 of the lower section 210. Additional sections may continue to be added to form a chain of sections as needed, and eventually the chain is wrapped around to form a closed loop by securing the two ends of the formed chain to each other. The closed loop should be sized to the particular body part in which the target region (i.e., the region that receives the TTFields therapy) is located. In some preferred embodiments, there are a total of two modules 200 providing one pair of electrode assemblies. In some preferred embodiments, there are a total of four modules 200 providing two pairs of electrode assemblies.

As noted, the electrode assemblies can be initially placed directly on the subject's body, in the desired therapeutic locations, using skin-compatible adhesives disposed on the fronts of the electrode assemblies. Alternatively, the electrode assemblies can be placed on individual stretchable band sections, or on a fully or partially formed chain of sections, according to an arrangement such that, when the chain of sections is placed on the subject's body part, it will surround the target region in the therapeutically required manner. Placement of the electrode assemblies on the band or band section can be performed by using an adhesive layer on the rears of the electrode assemblies, or by using attachment structures (provided on the band sections and/or on the electrode assemblies) for attaching the electrode assemblies to the stretchable band sections.

It is to be noted that in some situations, particularly when multiple bands are used to press against electrode assemblies, all of the multiple bands do not necessarily have to be stretchable. For example, in a chain of multiple bands/straps, it may be enough to have just one band that is stretchable in order to provide the capability of being able to stretch the chain to fit the particular dimensions and contours of the subject's body part. In some examples, all the straps/bands in the linked chain may be unstretchable, and variability in the length of the chain can then be achieved by adding band/strap sections to the chain or by providing at least one attachment overlap area large enough to control the overall loop size (by varying the amount of overlap). Thus, one or more straps and the plurality of electrode assemblies may be collectively shaped and dimensioned to completely surround a portion of the subject's body (e.g., without needing any of the straps to be stretchable).

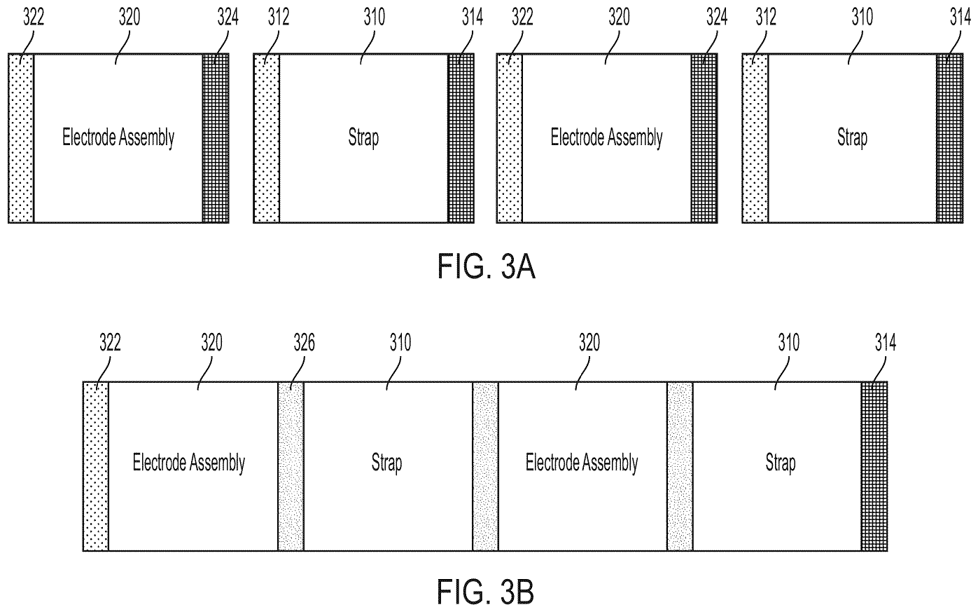

FIGS. 3A-B depict yet another approach for holding a plurality of electrode assemblies in place on a subject's body. In this approach, straps/bands (stretchable or otherwise) are interposed between the edges of adjacent electrode assemblies. When placed on a subject's body part, the bands hold the electrode assemblies at their desired positions relative to the target region being treated. When stretchable (e.g., elastic) bands are used, the bands can also act to press the electrode assemblies against the subject's body. Similar forces can be produced using unstretchable straps with fasteners (for example, hook and loop fasteners; or reversible reclosable fasteners) by forming a loop around the body part and using at least one overlap region to control a tight fit around the body part.

FIG. 3A shows two electrode assemblies 320 and two straps 310 prior to their attachment. Each of the electrode assemblies 320 has fasteners disposed on opposing edges of the electrode assembly. The fasteners could be, for example, hook and loop fasteners such as male Velcro® strips 322 extending transversely along one edge of the electrode assembly, and female Velcro® strips 324 extending transversely along the opposite edge of the electrode assembly (the specific sequence of male-female can be reversed, and the Velcro® strips do not necessarily need to occupy the entire side edges of the electrode assembly). In alternative embodiments, fasteners other than Velcro® (e.g., reversible reclosable fasteners, adhesives, and so on) may be used instead of or in addition to the Velcro® fasteners.

Positioned between each of the assemblies 320 are straps 310 (which may be stretchable or non-stretchable) that include fasteners that are compatible with the fasteners of the electrode assemblies 320. Thus, for example, each strap 310 can include a fastener such as a male Velcro® strip 312 that is connectable to the female Velcro® strip 324 of a respective neighboring electrode assembly 320. In addition, on the opposite side of each strap 310, there can be a female Velcro® strip 314 that is connectable to the male Velcro® strip 322 of a respective neighboring electrode assembly 320. It is noted that the attachment structures (e.g., 222 in FIGS. 2A-B) included with the straps 310 should be positioned on a surface of the straps 310 that directly faces, at attachment time, the portion of the electrode assembly 320 on which the electrode assemblies' attachment structures are disposed.

FIG. 3B shows one example of how the electrode assemblies and straps of FIG. 3A can be connected to each other to form a chain of components. In the example of FIG. 3B, the left most strap 310 links to the left most electrode assembly 320 by attaching the strap's male Velcro® strip 312 to the female Velcro® strip 324 of the left most electrode assembly 320. The attachments result in a joined (mated) Velcro® structure marked as strip 326. Similarly, the left most strap 310 attaches to the right most electrode assembly 320 by attaching the female Velcro® strip 314 of the left most strap 310 to the male Velcro® strip 322 of the right-most electrode assembly 320. Next, the female Velcro® strip 324 of the right most electrode assembly 320 is attached to the male Velcro® strip 312 of the right-most strap 310, to thus form a chain of four components, comprising alternating pairs of bands 310 and electrode assemblies 320 that are linked together. The chain of four components 310, 320 depicted in FIG. 3B can be further extended as needed to match the dimensions and contours of a subject's body part by adding additional pairs of electrode assemblies and straps. Those additional pairs would be attached to the linked sections of the chain in a manner similar to that discussed above for the first four components 310, 320 depicted in FIG. 3B. Eventually, the ends of the chain are linked to form a closed loop that surrounds the target region by attaching, in the example of FIGS. 3A-B, the female Velcro® strip 314 of the right-most strap 310 to the male Velcro® strip 322 of the left-most electrode assembly 320.

As noted, in some embodiments, the chain depicted in FIG. 3B may be formed using other types of fasteners (such as adhesive layers, different types of interlocking devices, etc.). Additionally, in some embodiments, the chain may include alternating groups of components other than the electrode assembly/strap pairs illustrated in FIGS. 3A-B. For example, each alternating group of components may include an electrode assembly (such as the electrode assembly 320) followed by two, or more, linked straps. In other embodiments, the chain may include multiple electrode assemblies directly linked to each other, followed by one or more straps such as the strap 310 illustrated in FIGS. 3A-B.

The particular arrangement and configuration of different chains would generally depend on the therapeutic requirements (e.g., how many electrode assemblies are needed) and the dimensions and contours of the body part on which the chain is to be placed. It is to be noted that in some embodiments, the entire chain of the electrode assemblies and straps may be formed ahead of deployment of the resultant chain on the subject's body part (with optimized locations of the electrode assemblies as determined below). The chain can be positioned around the body or body part and then a final join of two fasteners can form a loop around the body (part). In alternative embodiments, the electrode assemblies may first be placed on the body part and then linked using the straps to form a chain of alternating electrode assemblies and straps. In some embodiments, there are two straps and two electrode assemblies. In some embodiments, there are four straps and four electrode assemblies.

For a given subject undergoing TTFields treatment, it is possible to locate the appropriate optimized locations on the body for positioning the electrode assemblies (e.g., using Novotal or MaxPoint software, Novocure GmbH, following an MRI or CAT scan). If attachment structures 222 are positioned on each electrode assembly and the electrode assemblies are then positioned on the body, the appropriate placement of the counterpart attachment structures 114 on the belt can be arranged for a tightened fit that comfortably holds the electrode assemblies in place. This approach has the advantage of allowing reuse of the same stretchable belt or adjustable strap for a given subject (for example, by substituting new electrode assemblies with attachment structures 222 in place of recently-used ones that need to be replaced).

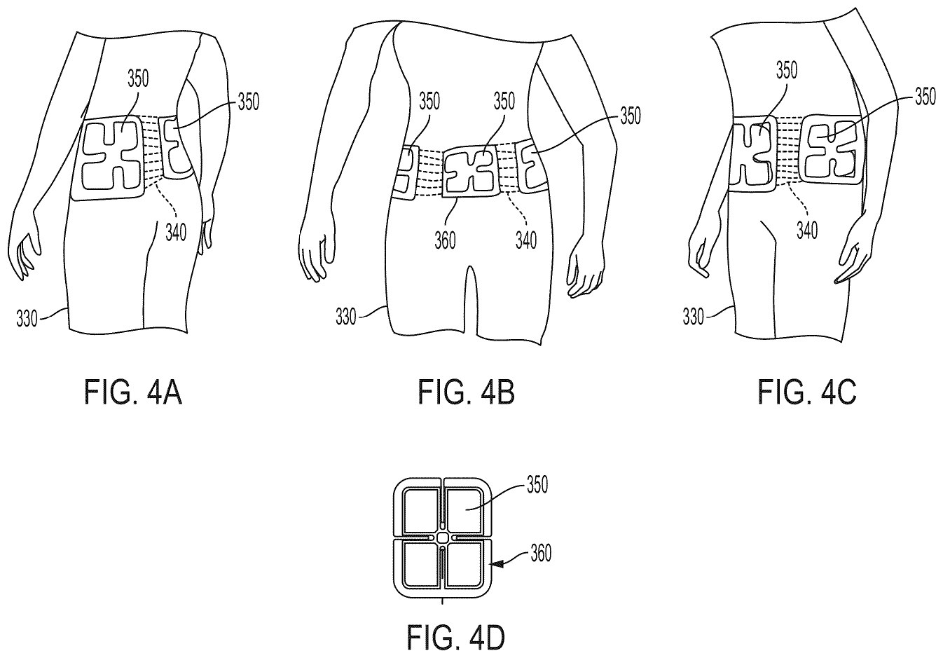

FIGS. 4A-D depict yet another approach for holding a plurality of electrode assemblies in place on a subject's body for treating a subject with TTFields therapy. More specifically, FIGS. 4A-C depict three different views of a subject 330 wearing an arrangement of four electrode assemblies 350 linked together by four band sections 340 around the subject's torso. In the example depicted in FIG. 4A-4D, each of the electrode assemblies has four electrode elements, as more particularly shown in FIG. 4D. But any of a variety of alternative configurations of electrode elements for each of the electrode assemblies 350 may be used. Each of the electrode assemblies can be secured to the body, in part, by using a band-aid type bandage such as the bandage 360 applied to the electrode assembly 350 of FIG. 4D (the bandage 360 is depicted as a frame within which an electrode assembly 350 is enclosed). As an alternative to an overlay bandage that covers the back face of the electrode assembly and extends beyond the outer perimeter of the electrode assembly, the perimeter of the electrode assembly may be framed (or partially framed) on the front-facing side with a layer of adhesive (or material, such as foam, having a layer of adhesive on the front-facing side).

In some embodiments, the electrode assemblies are positioned on the body at locations that are recommended by a software program (e.g., using the Novotal or MaxPoint software). The positioning can be accomplished, for example by first using a front-facing adhesive of a foam frame to hold the electrode assemblies against the subject's body with a relatively low degree of tack, and subsequently securing the positions of the electrode assemblies using the stretchable band(s) and/or straps described herein. Thus, for example, an apparatus for applying an alternating electric field to a target region in a subject's body can comprise (a) a plurality of electrode assemblies each of which has a front-facing perimeter at least partially framed by an exposed front-facing adhesive layer, with each electrode assembly configured to impose an alternating electric field in the target region of the subject's body; and (b) an adjustable strap reversibly coupled to a rear face of at least one of the electrode assemblies.

To further physically secure the electrode assemblies to the body, one or more bands, such as the bands 340, are attached to the sides of adjacent electrode assemblies (or to the band-aid type bandage applied to the assemblies). Similar to the bands described above, the connecting bands 340 may be stretchable or unstretchable, and, if unstretchable, may be adjustable by use of overlapping fasteners. In some embodiments, the bands linking the electrode assemblies around the body or body part are stretchable and may take the form of a material or fabric, such as an elasticated band. With reference to FIG. 4A-C, each linkage (band 340) between adjacent electrode assemblies 350 may comprise a single connecting band or multiple connecting bands (for example, multiple strips of elasticated material). In the case of a single stretchable band linking between adjacent electrode assemblies, the stretchable band may have a width similar to that of the electrode assemblies. For example, the stretchable band may have a width that matches the width of each electrode assembly, respectively, within ±5 cm. The matching tolerance can also be, for example, within ±2 cm, within ±1.5 cm, within ±1 cm, within ±8 mm, within ±7 mm, within ±6 mm, within ±5 mm, within ±4 mm, within ±3 mm, within ±2 mm, within ±1 mm, within ±0.8 mm, or within ±0.5 mm. For embodiments where the linkage between adjacent electrode assemblies is via multiple elasticated strips, the width of each strip may be less (even significantly less) than that of the electrode assemblies.

The stretchable bands that act as linkages between the electrode assemblies may be secured to the electrode assemblies by any means known in the art. For example, in some embodiments, the bands may have hook and loop material (e.g. Velcro®) tabs or strips attached to the ends of the bands, and these may be attached to other hook and loop material tabs or strips on the electrode assemblies 350 (or overlay bandages 360 or similar support substrates). In other embodiments, the bands may have tabs or strips with a layer of adhesive at each connecting end and these may be used to attach to the neighboring electrode assembly. In another embodiment, one end of the band 340 may be sewn onto the edge of an electrode assembly 350 or its overlay bandage 360 or similar support substrate. Alternatively, both ends of the linking band 340 may be sewn onto respective edges of two adjacent electrode assemblies 350 (or its overlay bandages 360 or similar support substrates). Linkages may be connected around the body or body part to form a closed loop, as shown in FIGS. 4A-C. In forming a closed loop, for the sake of convenience for the subject, it may be preferred to have at least one connection point between adjacent electrode assemblies with a non-permanent closing linkage, such as a hook and loop fastener, other repositionable fastener, or adhesive area (as opposed to sewn connection points all around the loop). Other fastener or garment closure mechanisms may also be incorporated, especially as the final closure point. For example, common bra clasps/closures may be used, such as hook and eye closures, ring, slide and hook closures, etc.

In the example depicted in FIGS. 4A-C, each electrode assembly 350 is connected to both of its neighbors by multiple thin bands that are vertically stacked. Collectively, the alternating sets of bands and electrode assemblies are linked together to form a closed loop.

Examples of thin bands that may be used to connect electrode assemblies in the embodiments of FIGS. 4A-C include reclosable fastener bands such as, for example, 3M™ hook-and-loop reclosable fasteners, hook-and-loop Velcro® fasteners, or the 3M™ Dual Lock® reclosable fasteners, or the ALFA-LOK™ Velcro® fasteners, which are configured to fasten two layers of interlocking materials to each other. Reclosable fastener bands are implemented as strips comprising layers of interlocking fasteners (hook-and-loop fasteners, mushroom-head fasteners, etc.) The reclosable fasteners have interlocking surfaces designed to mate (to form a 2-layer band). Optionally, each of the exterior surfaces of the 2-layer band includes an adhesive layer covered with a peelable material (release liner) that is removed prior to deployment of the bands. In some embodiments, instead of using an adhesive, the exterior layers may include a mesh fabric that make the 1-layer interlocking band more repositionable than they would be with adhesive layers.

In operation, the two layers of strips of interlocking bands can be separated, and a first separated 1-layer interlocking band is then attached, at an end area of the first 1-layer band to a first electrode assembly 350 (or to the overlay bandage 360) by pressing the adhesive layer described above (after peeling the release liner to expose the adhesive layer) to an overlap area on the first electrode assembly (or overlay bandage). The overlap area can be either the entirety of or a portion of the rear surface of the first electrode assembly (or its overlay bandage). The remainder of the separated 1-layer band can be extended around the subject's body part, and have its other end either connected to another, second, electrode assembly (or its overlay bandage) (e.g., to a portion of the rear surface of the other electrode assembly or overlay bandage), or having the other end area of the 1-layer band simply secured to the skin on the body part (i.e., with the adhesive layer on the separated 1-layer interlocking band pressed against the skin).

The second 1-layer interlocking band can be similarly attached to the second electrode assembly or its overlay bandage (e.g., to the entirety, or a portion of the second electrode assembly or its overlay bandage) by pressing an adhesive layer (after peeling the release liner to expose the adhesive layer) disposed on the back surface of the second 1-layer interlocking layer to the second electrode assembly or overlay bandage. The remainder of the second 1-layer band may be secured to the subject's skin by, for example, pressing the adhesive layer of the second 1-layer band to the subject's skin. The end section of the second separated 1-layer band (i.e., the end not attached to the second assembly array) can be linked directly back to the first electrode assembly and fastened to the first 1-layer band by interlocking the two layers, or wrapped around the body to the first electrode assembly to interlock with the first separated 1-layer band and form a closed loop (if, for example, the chain includes just two electrode assemblies). Alternatively, the end section of the second separated 1-layer band can be extended to the next electrode assembly (thus extending the chain).

To further illustrate, consider the example of a chain that includes just two electrode assemblies using a 2-layer hook-and-loop reclosable fastener band. One 1-layer interlocking band (e.g., the hook layer, separated from the 2-layer reclosable fastener band) may be positioned on the front torso, and the other 1-layer interlocking band may be positioned on the back torso. The back side of the hook layer can be placed on an overlap region on the side of the first electrode assembly, and the rest of the 1-layer hook band can then be extended around the side of the body, optionally securing it to the subject's body with the adhesive layer that is on the back surface of the 1-layer hook band. Then, from the second electrode assembly, the back side adhesive of the 1-layer loop band (separated from the hook band with which it formed the 2-layer reclosable fastener band) is attached to the near edge of the second electrode assembly. The rest of the 1-layer loop band is extended around the body (optionally so that the adhesive material sticks to the body), and then is secured to the 1-layer hook band attached to the first electrode assembly. This same procedure is then repeated in the other direction (i.e., going the other way around the body).

A similar procedure can be used to link four electrode assemblies around the torso as depicted in FIGS. 4A-C. Positioning four electrode assemblies, for example, with one pair positioned in front and in back of the torso, and the other pair positioned on the two sides of the torso, can facilitate treatment of a cancer in the torso, including but not limited to pancreatic cancer, stomach cancer, intestinal cancer, lung cancer (e.g., non-small cell lung cancer, NSCLC).

Although FIGS. 1-4 depict four specific approaches for using bands to hold electrode assemblies against the subject's body, a variety of alternative approaches that rely on bands can also be used to hold the electrode assemblies against the subject body. We shall now turn to a description of the electrode elements themselves.

FIG. 5 depicts one example of an electrode assembly that can be held against the subject's body using one or more bands. More specifically, FIG. 5 is a cross sectional representation of an embodiment of an electrode assembly 400 that includes two electrode elements E1 and E2, and which incorporates a sheet of graphite 410 into the electrode assembly (electrode assemblies may include only a single electrode element, or may include more than two electrode elements). The use of the graphite sheet spreads both heat and current out in directions that are parallel to the front face of the sheet, which eliminates or at least minimizes hot spots on the electrode assembly. Further details regarding the embodiments of the electrode assembly of FIG. 5 are provided in US 2023/0043071, entitled “Electrode Assembly for Applying Tumor Treating Fields (TTFields) that Include a Sheet of Graphite,” the content of which is incorporated herein by reference in its entirety.

Briefly, the electrode assembly 400 includes a sheet of graphite 410 having a front face (facing towards the subject's skin in FIG. 5) and a rear face. Examples of suitable forms of graphite include synthetic graphite, such as pyrolytic graphite (including, but not limited to, Pyrolytic Graphite Sheet (PGS), available from Panasonic Industry, Kadoma, Osaka, Japan), other forms of synthetic graphite, including but not limited to, graphite foil made from compressed high purity exfoliated mineral graphite (including, but not limited to, that supplied by MinGraph® 2010A Flexible Graphite, available from Mineral Seal Corp., Tucson, Arizona, USA), graphitized polymer film, e.g., graphitized polyimide film, (including, but not limited to, that supplied by Kaneka Corp., Moka, Tochigi, Japan), isotropic graphite (including but not limited to isotropic graphite grade G330 available from Tokai Carbon Europe, Oldbury, UK), or double-sided carbon tape for scanning electron microscopy, available from Fisher Scientific, a unit of Thermo Fisher Scientific, Hampton, N.H., USA).

The electrode assembly 400 further includes at least one layer of biocompatible conductive material 420 disposed on the front face of the sheet 410. The at least one layer of conductive material 420 is configured to ensure good electrical contact between the device and the body. In some embodiments, the at least one layer of material 420 should cover the entire front face of the sheet of pyrolytic graphite 410, but may be larger in size than the sheet of pyrolytic graphite 410. Although not shown in FIG. 5, the at least one layer of conductive material 420 may be or may include a layer of hydrogel, which may have a thickness between 50 and 2000 μm (and more particularly may have a thickness from 100 to 1000 μm, or even 300 to 500 μm). In some embodiments, the at least one layer of conductive material 420 may be a single layer of non-hydrogel biocompatible conductive adhesive such as the developmental product FLX068983—FLEXcon® OMNI-WAVE™ TT 200 BLACK H-502 150 POLY H-9 44PP-8 from FLEXcon, Spencer, MA, USA, or other such OMNI-WAVE products from FLEXcon; or ARcare® 8006 electrically conductive adhesive composition manufactured and sold by Adhesives Research, Inc. (Glen Rock, PA, USA). Such non-hydrogel conductive adhesives may include a waterless polymer with adhesive properties and carbon particles, powder, fibers, flakes or nanotubes. The adhesive polymer may be, for example, an acrylic polymer or a silicone polymer, or combination thereof, which may be available as acrylic- or silicone-based carbon-filled adhesive tapes. The adhesive may additionally include one or more conductive polymer (such as, for example, polyaniline (PANI) or poly (3,4-ethylenedioxythiophene (PEDOT), or others known in the art). The conductive filler in the at least one layer of conductive material 420 should be non-metallic. In these embodiments, the biocompatible conductive adhesive may have a thickness between 10 and 2,000 μm, 20 to 1000 μm, or 30 to 400 μm.

With continued reference to FIG. 5, the electrode assembly 400 further includes a first electrode element E1 positioned behind the graphite sheet 410. The first electrode element E1 includes a first front face disposed in electrical contact with the rear face of the sheet 410. In some embodiments, the first electrode element E1 may include a first layer of dielectric (e.g., ceramic) material 432 having a front face and a rear face, and a first layer of metal 430 disposed on the rear face of the first layer of dielectric material 432. When present, the front face of the first layer of dielectric material 432 is the first front face of the first electrode element E1. It is to be noted that a variety of suitable dielectric materials, other than ceramic, may be used. Examples include a polymer layer that has a dielectric constant of at least 10, or another material having a dielectric constant of at least 10. In some embodiments, the layer of dielectric material 432 can have a dielectric constant ranging from 10 to 50,000.

In various examples, the electrode assembly 400 may further include a first rear layer of conductive material 440 positioned between the first front face of the first electrode element E1 (e.g., the front face of the first layer of dielectric material, if present) and the rear face of the sheet 410. The first rear layer of conductive material 440 facilitates the electrical contact between the first front face of the first electrode element E1 and the rear face of the sheet 410. In some embodiments, the rear layer of conductive material 440 may be a layer of hydrogel, but different conductive materials (e.g., conductive grease, conductive adhesive, conductive tape, conductive composite, etc.) could be used instead.

As noted, and as depicted in FIG. 5, the electrode assembly 400 includes a second electrode element E2 positioned behind the graphite sheet 410. The second electrode element E2 has a second front face disposed in electrical contact with the rear face of the sheet 410. The two electrode elements E1, E2 in FIG. 5 typically have the same structure, and thus, the second electrode element E2 may include a second layer of dielectric (e.g., ceramic) material, such as the material 432 of the electrode E1, having a front face and a rear face, and a second layer of metal 430 disposed on the rear face of the second layer of dielectric material 432.

The first rear layer of conductive material 440 is positioned between the second front face of the second electrode element E2 (e.g., the front face of the second layer of dielectric material 432, if present) and the rear face of the graphite sheet 410. The first rear layer of conductive material 440 facilitates the electrical contact between the second front face of the second electrode element E2 and the rear face of the sheet 410. The conductive material 440, disposed at the front surface of the ceramic layer 432, may be a layer of hydrogel, but in alternative embodiments, a different conductive material may be used (e.g., conductive grease, conductive adhesive including the non-hydrogel conductive adhesive, conductive tape, conductive composite, etc.).

The metal layers 430 of all of the electrode elements (i.e., E1 and E2 in the illustrated embodiment), may be wired together (e.g., using wires, traces on a flex circuit, etc.) to a lead 450. The lead 450 supplies an AC voltage from an AC voltage generator (not shown in FIG. 5, but shown in FIG. 7) to the electrode elements to generate the TTFields when the electrode assembly 400 is affixed to the subject's body for treatment. Optionally, the electrode assembly 400 includes a flexible self-adhesive backing 460 (or overlay bandage) configured to support the sheet 410, the first electrode element E1 (and any other electrode elements present in the electrode assembly), and the at least one layer of conductive material 420 so that the front surface of the at least one layer of conductive material 420 can be positioned against the subject's skin.

It is to be noted that in some embodiments the electrode assembly 400 may lack the dielectric layer 432 of the electrode elements E1 and/or E2 (i.e., the electrode assembly may be implemented without a dielectric layer). In some embodiments, the electrode assembly 400 may be implemented with the first front face of the first electrode element E1 being positioned in direct contact with the rear face of the graphite sheet 410, instead of being electrically connected via an intervening layer of conductive material (such as the conductive material 440 of FIG. 5).

The implementations of an electrode assembly comprising a pyrolytic graphite sheet may include different variations that may be beneficial for its operation under different circumstances. However, the base structure/implementation of the electrode assembly of FIG. 5 (on its own, or when used in conjunction with the apparatus illustrated in FIGS. 1-4) includes at least one conductive pad, and a sheet of graphite positioned in front of the at least one conductive pad. In some embodiments, a layer of conductive adhesive or a layer of conductive hydrogel may be positioned against both the front side of the sheet (layer) of graphite and the rear side of the sheet of graphite.

FIG. 6 depicts another example of an electrode assembly 500 that can be held against the subject's body using one or more bands e.g., as described above in connection with FIGS. 1-4. The example electrode assembly 500 comprises two discrete subassemblies 510, 550 that are removably connectable to each other. This separable configuration for the electrode assembly allows, for example, one of the subassemblies (e.g., either the top sub-assembly that is radially farther away from the subject's body part, or the bottom/front sub-assembly that comes in contact with the subject skin's) to be re-used.

During use, the first and second subassemblies 510 and 550 of electrode assembly 500 are pressed against each other and are in intimate contact. However, to facilitate explanation of the electrode assembly configuration and operation, FIG. 6 depicts the two sub-assemblies in a separated configuration, more clearly showing the boundaries between the sub-assemblies. The first subassembly 510 (referred to as a top sub-assembly or the rear sub-assembly or electrode subassembly) includes a first layer of conductive polymer 520 positioned at the front of the first subassembly in a central region of the first subassembly, so that a front surface of the first layer of conductive polymer 520 may serve as a front surface of the first subassembly 510 in the central region of the first subassembly. The first layer of conductive polymer 520 may have an example area of at least 10 cm². Examples of suitable materials for the first layer of conductive polymer 520 include conductive versions of polymers including, but not limited to silicone, silicone rubber, natural rubber, poly cisisoprene, polyisobutylene, chloroprene, cis-polybutadiene, styrene-butadiene, styreneacrylonitrile-butadiene, polyurethane, EPDM, EVA polymers, and perfluoropolymers. Such polymers can be modified to produce conductive versions of the polymers by infusing them with conductive particles, such as, for example, metal particles or carbon particles. Carbon particles may include, for example, carbon flakes, carbon granules, carbon fibers, carbon black powder, graphite powder, carbon nanotubes, carbon nanowires, and the like.

The first subassembly 510 also includes at least one electrode element 512 positioned between the first layer of conductive polymer 520 and the rear of the first subassembly 510. Each of the electrode elements 512 has a front surface. In the embodiment depicted in FIG. 6, the electrode elements 512 are metal (e.g., copper) pads that are disposed on the front face of a first PCB 530. When more than one metal pad 512 is included (as depicted in FIG. 6), all the metal pads 512 can be connected by metal traces (not shown in FIG. 6). It is to be noted that as used herein, the term “PCB” refers to a printed circuit board, and this term encompasses rigid PCBs (e.g., with copper traces on a rigid epoxy board), flex circuits (e.g., with copper traces on a flexible polyimide substrate), and printed circuits made by printing a conductive ink on a flexible substrate, etc.

At least one intermediate layer of material is positioned between the front surface of each of the electrode elements 512 and a rear surface of the first layer of conductive polymer 520. For example, in FIG. 6, the at least one intermediate layer of material comprises a layer of insulating material 524 with a dielectric constant of at least 10, and a first layer of conductive adhesive 522 disposed on, and positioned in front of, the layer of insulating material 524. The first layer of conductive polymer 520 is disposed on, and positioned in front of, the first layer of conductive adhesive 522. And because the layer of insulating material 524 has a high dielectric constant, the layer of insulating material 524 and the first layer of conductive adhesive 522 will collectively capacitively couple each of the electrode elements 512 to the first layer of conductive polymer 520.

Examples of suitable materials for the first layer of conductive adhesive 522 include, but are not limited to, the OMNI-WAVE™ adhesive compositions manufactured and sold by FLEXCON® (Spencer, MA, USA), such as the developmental product FLX068983—FLEXcon® OMNI-WAVE™ TT 200 BLACK H-502 150 POLY H-9 44PP-8; and the adhesives from ADHESIVE RESEARCH, such as ARcare® 8006 electrically conductive adhesive composition manufactured and sold by Adhesives Research, Inc. (Glen Rock, PA, USA). Alternatively, Electrically Conductive Adhesive Transfer Tape 9712 or Electrically Conductive Adhesive Transfer Tape 9713 (both manufactured by 3M) may also be used. Examples of suitable materials for the layer of insulating material 524 include, but are not limited to, at least one of Poly (VDF-TrFE-CTFE), Poly (VDF-TrFE-CFE), and Poly (VDF-TrFE-CFE-CTFE), and/or ceramic nanoparticles mixed into at least one of Poly (VDF-TrFE), P(VDF-HFP), PVDF.

In some embodiments, the layer of insulating material 524 may be omitted, in which case the at least one intermediate layer of material will comprise just the first layer of conductive adhesive 522, which, in that case, would be disposed on, and positioned in front of, the electrode elements 512. In this embodiment, the first layer of conductive polymer 520 is disposed on, and positioned in front of, the first layer of conductive adhesive 522. And because the layer of insulating material 524 is not present, the at least one intermediate layer of material (i.e., the first layer of conductive adhesive 522) will conductively couple each of the electrode elements 512 to the first layer of conductive polymer 520.

In some variations, the first (top/rear) subassembly 510 may also include a plurality of conductive terminals (such as the terminals X5 and X6 shown in FIG. 6), positioned at the front of the first subassembly in a peripheral region of the first subassembly. Conductive traces or wires are provided between each of the conductive terminals and a respective pin of a connector (not shown) to provide the controller (discussed herein with respect to FIG. 7) with access to the conductive terminals.

With continued reference to FIG. 6, the second subassembly 550 includes a second layer of conductive adhesive 560 positioned at the rear of the second subassembly 550, so that a rear surface of the second layer of the conductive adhesive 560 serves as a rear surface of the second subassembly 550. The second layer of conductive adhesive 560 is shaped and dimensioned to overlap both the central region of the first subassembly 510 (i.e., the region that corresponds to the first layer of conductive polymer 520) and the peripheral region of the first subassembly 510 (i.e., the region that corresponds to the second PCB 540). A layer of graphite 570 is disposed on, and positioned in front of, the second layer of conductive adhesive 560. A third layer of conductive material 580 is disposed on, and positioned in front of, the layer of graphite 570. The third layer of conductive material 580 is configured to adhere to skin and can be a biocompatible conductive adhesive.

Examples of suitable materials for the second layer of conductive adhesive 560 include, but are not limited to, materials similar to those described above for the first layer of conductive adhesive 522. Examples of suitable materials for the layer of graphite 570 include, but are not limited to the various materials discussed with respect to the pyrolytic graphite sheet discussed in relation to FIG. 5. In alternative embodiments, instead of using a layer of graphite 570, a layer of another anisotropic material may be used. Examples of suitable materials for the third layer of conductive material 580 include any suitable biocompatible adhesive that is designed to be removably affixed to a person's skin. These may also include, but are not limited to, the same materials described above for the first layer of conductive adhesive 522 discussed in relation to the first subassembly 510.

As illustrated in FIG. 6, a stretchable band 502, which may be similar in configuration to the stretchable bands of FIGS. 1-4, including the single band that forms a loop when its two ends are attached, or the multiple band sections (whether stretchable or not) that are linked to form a chain configuration. In some examples, the band 502 may be a flexible backing (e.g., a bandage-like backing) that may be positioned behind the first and second PCBs 530, 540, and this flexible backing is configured to support both of those PCBs. A portion of such a backing (e.g., band 502) may extend laterally beyond the second PCB 540, and the front of this portion may optionally be covered with a biocompatible adhesive that adheres to skin. This portion of the band 502 helps hold the first and second subassemblies 510 and 550 against the subject's skin.

When the first subassembly 510 is in contact with the second subassembly 550 (i.e., by moving those two subassemblies towards each other until the gap between those two is eliminated), the front surface of the first layer of conductive polymer 520 will be positioned against the rear surface of the second layer of conductive adhesive 560.

Thus, in various embodiments, electrode assemblies that may be used in conjunction with the one or more bands (stretchable or unstretchable) of the apparatus described herein may include an example electrode assembly that includes a first subassembly that is removably connectable to a second subassembly, with the first subassembly including a first conductive layer (e.g., the conductive polymer 520) with a front surface facing toward the subject's body and a rear surface facing away from the subject's body, and with the front surface of the first conductive layer being a front surface of the first subassembly. The first subassembly may further include a dielectric layer 524 positioned between a rear of the first subassembly and the rear surface of the first conductive layer, and one or more electrode elements positioned between the rear of the first subassembly and the dielectric layer.

It will be appreciated that many other types of electrode assemblies, configured to apply TTFields, and structured for use with the apparatus described herein (e.g., one or more stretchable bands configured to press a plurality of electrode assemblies against respective locations of the subject's body so that the target region will be positioned between the plurality of electrode assemblies) may be used in embodiments of the present disclosure. An additional, non-limiting example, of one such electrode assembly type is an electrode assembly with a flexible polymer layer, as more particularly described in U.S. Publication No. 2021/0402179, entitled “Flexible Transducer Arrays with a Polymer Insulating Layer for Applying Tumor Treating Fields (TTFields),” the content of which is incorporated herein by reference in its entirety.

It is important to note that the electrode assemblies depicted in FIGS. 5 and 6 are not the only types of electrode assemblies that may be held against the subject's body using one or more bands. To the contrary, any of a variety of alternative approaches for constructing the electrode assemblies may be used.

FIG. 7 depicts a system 600 that applies TTFields to target regions within a subject's body using a plurality of electrode assemblies that are held against the subject's body using one or more bands (including but not limited to the bands described above in connection with FIGS. 1-4). Note that the electrode assemblies depicted in FIG. 7 are two-part electrode assemblies similar to the ones described above in connection with FIG. 6. But other types of electrode assemblies can be used in place of the depicted two-part electrode assemblies.

In FIG. 7, the first electrode assembly includes sub-assembly 601 removably connected to sub-assembly 602 that is positioned at one side of a target region in the subject's body, and a second electrode assembly that includes a sub-assembly 603 removably connected to sub-assembly 604 which is positioned at the other side of the target region in the subject's body such that the target region is located between the two assemblies. For example, the target region may be located in the subject's head or in the subject's torso.

The system 600 further includes one or more bands 640 (e.g., stretchable bands) configured to press the electrode assemblies (in this case the combination of removably connected sub-assemblies pairing 601 and 602, and 603 and 604) against the subject's body part, and maintain the electrode assemblies at substantially fixed positions relative to the subject's target region.

While FIG. 7 depicts a single stretchable band 640 in a closed loop configuration, any of a variety of alternative band configurations (including but not limited to any of the embodiments described above in connection with FIGS. 1-3) can be used to hold the electrode assemblies against the subject's body. These alternatives include, but are not limited to, using multiple modules attached to each other to form a linked configuration (e.g., as described above in connection with FIGS. 2A-B), or by alternating (interleaving) electrode assemblies and bands or straps (e.g., attaching a band to the adjacent edges of two neighboring electrode assemblies), as described above in connection with FIGS. 3 and 4.

In the example of FIG. 7, the single stretchable band forms the closed loop by attaching attachment structures (e.g., hook-and-loop, reversible reclosable fasteners, etc.) positioned at ends of the stretchable band. The attachment area of the stretchable band 640 is represented schematically by area 642 of the band 640.

Positioning of the electrode assemblies at particular locations on a body part of the subject (such locations may have been computed a priori, and determined to be optimal, or near-optimal, locations for applying TTFields) can be performed in one of several ways. For example, the electrode assemblies may be placed at the pre-determined locations on the body part using an adhesive (hydrogel, or some other conductive adhesive corresponding, for example, to the conductive adhesive layer 580 in FIG. 6). In the case of electrode assemblies that each have two or more removably connected sub-assemblies, a front sub-assembly of each of the assemblies may first be placed at the pre-determined locations, and once placed, the remaining top/rear sub-assemblies are attached to the front sub-assemblies, followed by placement of the one or more bands connected to the electrode assemblies at locations that press the electrode assemblies against the desired locations on the subject's body part.

Alternatively, placement of the electrode assemblies can be performed by first placing the electrode assemblies at pre-determined locations on the one or more stretchable bands, e.g., using attachment structures such as interlocking fastener patches secured to the rear portions of the electrode assemblies and to the contact surface of the one or more stretchable bands, or using adhesives to connect the rear portions of the electrode assemblies to the pre-determined locations on the one or more stretchable bands. In situations involving the use of multiple sub-assemblies to form the electrode assemblies, the rear portions may be placed on the one or more stretchable bands, and navigated to connect to their counterpart front portion sub-assemblies (e.g., via an adhesive layer, such as the conductive adhesive layer 560 of FIG. 6).

It is to be noted that the preparation of a subject for a treatment session, i.e., the mounting of electrode assemblies and stretchable bands/straps, may be facilitated by using, for every patient, pre-arranged kits with the needed materials. Such kits (arranged by a lab technician ahead of performing the treatment, or available in a commercially acquired kit) contain the materials needed to prepare a subject for TTFields treatment. Thus, in some embodiments, a kit for use in applying an alternating electric field to a target region in a subject's body is provided that includes a plurality of electrode assemblies, each of which may have a respective width and each of which is configured to impose an alternating electric field in the target region of the subject's body, and one or more stretchable bands and/or straps connecting two adjacent electrode assemblies, or connecting a series of electrode assemblies, and collectively shaped and dimensioned to press the plurality of electrode assemblies against respective locations of the subject's body so that the target region will be positioned between the plurality of electrode assemblies. In some embodiments, the kit may also include a voltage generator or at least the wiring required to connect the electrode assemblies to the voltage source and/or to a controller.

As further illustrated in FIG. 7, to impose alternating electric fields in the target region, the AC voltage generator 620 applies an AC voltage (e.g., between 50 kHz and 5 MHz, or 75-300 kHz) between the electrode elements (e.g., the metal pads) of the first electrode assembly (comprising sub-assemblies 601 and 602 in the illustrated example of FIG. 7) and the electrode elements of the second electrode assembly (comprising sub-assemblies 603 and 604). The AC signal from the AC voltage generator 620 arrives at each of the first and second electrode assemblies via a set of cables that route the AC signals (e.g., via metal traces, such as metal trace 450 depicted in relation to electrode assembly 400 of FIG. 5, or via other intervening components that are not shown in the figures) to, for example, the metal pads 512 of the first subassembly (namely, the rear sub-assembly such as the sub-assembly 510 shown in FIG. 6).

Because the first and second subassemblies of each electrode assembly are connected to each other, an AC signal that is applied to the metal pads/electrode contacts 512 of opposing electrode assemblies will, in example embodiments of the electrode assembly based on FIG. 6, be capacitively coupled across the layer of insulating material 524 in each of the electrode assemblies, and will subsequently pass through all of the conductive layers in front of the layer of insulating material 524 (i.e., the first layer of conductive adhesive 522, the first layer of conductive polymer 520, the second layer of conductive adhesive 560, the layer of graphite 570, and the third layer of conductive material 580). Because the third layer of conductive material 580 of each of the opposing electrode assemblies (601 and 602, vs. 603 and 604) is in contact with the subject's skin on opposite sides of the target region, alternating electric fields will be induced through the target region.

The AC voltage generator 620, and the system 600 in general, may be controlled by a controller 630 (e.g., a processor-based controller). The controller 630 may dynamically control characteristics of the voltage delivered by the AC generator 620 (including voltage amplitude and frequency) based on pre-determined power delivery profiles (e.g., in accordance with a medically prescribed therapy plan), and further based on operating conditions. For example, the controller 630 may be in electrical communication with various sensors deployed in the system 600, and may be configured to receive measurement data from such sensors. For instance, the controller 630 may use temperature measurements (e.g., measured by thermistors) to control the amplitude of the voltage to be applied via the first and second electrode assemblies in order to maintain temperatures below a safety threshold (e.g., 41° C.). This may be accomplished, for example, by measuring temperatures of the electrode elements, and controlling the alternating voltage based on the measured temperatures.

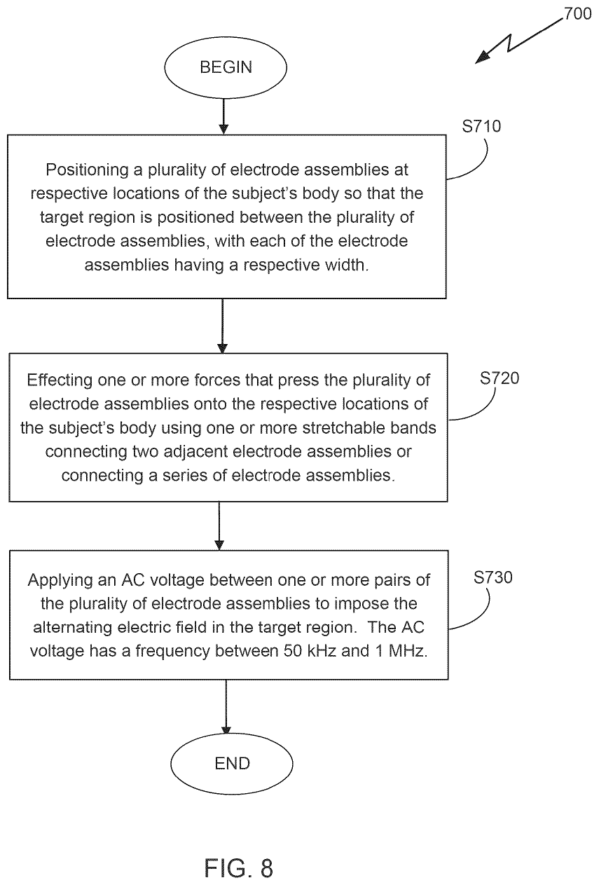

With reference next to FIG. 8, a flowchart of an example procedure 700 for applying an alternating electric field to a target region in a subject's body is shown. The procedure 700 includes positioning (in step S710) a plurality of electrode assemblies at respective locations of the subject's body so that the target region is positioned between the plurality of electrode assemblies. Each of the electrode assemblies may have a respective width. The procedure 700 further includes effecting (step S720) one or more forces that press the plurality of electrode assemblies onto the respective locations of the subject's body using one or more stretchable bands connecting two adjacent electrode assemblies or connecting a series of electrode assemblies, and applying (step S730) an AC voltage between one or more pairs of the plurality of electrode assemblies to impose the alternating electric field in the target region. The AC voltage has a frequency between 50 kHz and 1 MHz. In some examples, the AC voltage may have a frequency range of 100 kHz and 300 kHz.

In various examples, the one or more stretchable bands may have a width that matches the width of each electrode assembly, respectively, within ±5 cm. The matching tolerance can also be, for example, within ±2 cm, within ±1.5 cm, within ±1 cm, within ±8 mm, within ±7 mm, within ±6 mm, within ±5 mm, within ±4 mm, within ±3 mm, within ±2 mm, within ±1 mm, within ±0.8 mm, or within ±0.5 mm.

In some embodiments, at least one of the one or more stretchable bands may be positioned both over at least one of the plurality of electrode assemblies and between at least one adjacent pair of electrode assemblies. In some other embodiments, each of the one or more stretchable bands may be positioned only between adjacent pairs of electrode assemblies such that each pair of electrode assemblies are connected by a stretchable band. At least one of the one or more stretchable bands may be positioned only between adjacent pairs of electrode assemblies.

Headings are provided for convenience only and are not to be construed to limit the invention in any manner. Embodiments illustrated under any heading or in any portion of the disclosure may be combined with embodiments illustrated under the same or any other heading or other portion of the disclosure. Any combination of the elements described herein in all possible variations thereof is encompassed by the invention unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an alternating electric field to a target region in a subject's body, the apparatus comprising:
a plurality of electrode assemblies, each of which has a respective width and each of which is configured to impose an alternating electric field in the target region of the subject's body; and
one or more stretchable bands connected to the plurality of electrode assemblies, the one or more stretchable bands collectively shaped and dimensioned to press the plurality of electrode assemblies against respective locations of the subject's body so that the target region will be positioned between the plurality of electrode assemblies.

2. The apparatus of claim 1, wherein the one or more stretchable bands, connected to the plurality of electrode assemblies at corresponding locations on the one or more stretchable bands, have widths at the corresponding locations that match the respective ones of the plurality of electrode assemblies within ±1 cm.

3. The apparatus of claim 1, wherein at least one of the one or more stretchable bands is positioned both over at least one of the plurality of electrode assemblies and between at least one adjacent pair of electrode assemblies.

4. The apparatus of claim 1, wherein the one or more stretchable bands is a single stretchable band and is configured to form a closed loop when a first end of the single stretchable band is attached to a second end of the single stretchable band.

5. The apparatus of claim 4, wherein an unstretched length of the closed loop is adjustable.

6. The apparatus of claim 4, wherein the single stretchable band has a first end and a second end,
wherein the apparatus further comprises a first fastener positioned at the first end of the single stretchable band and a second fastener positioned at the second end of the single stretchable band, and
wherein the first and second fasteners are configured and positioned to hold the single stretchable band in a closed loop shape when the first and second fasteners mate with each other.

7. The apparatus of claim 6, wherein the first and second fasteners are hook-and-loop fasteners or are reversible reclosable fasteners.

8. The apparatus of claim 4, further comprising a plurality of attachment structures, each of which is disposed at a respective location on the single stretchable band, wherein each of the attachment structures is attached to a respective one of the electrode assemblies.

9. The apparatus of claim 1, wherein at least one of the one or more stretchable bands is positioned only between adjacent pairs of electrode assemblies.

10. The apparatus of claim 9, wherein the at least one of the one or more stretchable bands is connected to at least one of the plurality of electrode assemblies via a hook-and-loop fastener or by a reversible reclosable fastener.

11. The apparatus of claim 9, wherein the at least one of the one or more stretchable bands is sewn onto a support of at least one of the plurality of electrode assemblies.

12. The apparatus of claim 9, wherein each of the one or more stretchable bands are positioned only between adjacent pairs of electrode assemblies such that each pair of electrode assemblies are connected by a stretchable band.

13. The apparatus of claim 12, wherein alternating pairs of stretchable bands and electrode assemblies are linked together into a closed loop.

14. The apparatus of claim 13, wherein an unstretched length of the closed loop is adjustable.

15. The apparatus of claim 1, wherein each of the electrode assemblies comprises:
at least one conductive pad; and
a layer of graphite positioned in front of the at least one conductive pad.

16. An apparatus for applying an alternating electric field to a target region in a subject's body, the apparatus comprising:
a plurality of electrode assemblies, each of which is configured to impose an alternating electric field in the target region of the subject's body; and
one or more straps collectively shaped and dimensioned to hold the plurality of electrode assemblies against respective locations of the subject's body so that the target region will be positioned between the plurality of electrode assemblies,
wherein the one or more straps and the plurality of electrode assemblies are collectively shaped and dimensioned to completely surround a portion of the subject's body.

17. The apparatus of claim 16, wherein the one or more straps and the plurality of electrode assemblies are collectively held together using one or more hook-and-loop fasteners and/or reversible reclosable fasteners.

18. The apparatus of claim 17 comprising a single strap configured to form a closed loop around the subject's body and hold the electrode assemblies against the respective locations of the subject's body.

19. The apparatus of claim 16, wherein at least one of the one or more straps is positioned both over at least one of the plurality of electrode assemblies and between at least one adjacent pair of electrode assemblies.

20. The apparatus of claim 16, wherein each of the one or more straps are positioned only between adjacent pairs of electrode assemblies such that each pair of electrode assemblies are connected by a strap, and wherein the apparatus comprises two electrode assemblies and two straps, or comprises four electrode assemblies and four straps, in each case configured to form a closed loop around the subject's body.
